(19) United States
(12) Patent Application Publication
(10) Pub. No.: US 2009/0299063 A1
(43) Pub. Date: Dec. 3, 2009

(54) Inhibitors for Extracellular Signal-Regulated Kinase Docking Domains and Uses Therefor

(76) Inventors: Paul Shapiro, Baltimore, MD (US); Alexander D. MacKerell, JR., Baltimore, MD (US)

Correspondence Address: Benjamin Aaron Adler;Adler & Associates, 8011 Candle Lane, Houston, TX, 77071, US

(21) Appl. No.: 11/887,611

(22) PCT Filed: Mar. 29, 2006
(86) PCT No.: PCT/US2006/011536

§ 371 Date: Apr. 30, 2009

Related U.S. Application Data
(60) Provisional application No. 60/666,206, filed on Mar. 29, 2005.

(51) Int. Cl. C07D 413/12; C07D 417/04; C07D 265/30; C07D 241/04

(52) U.S. Cl. 544/175; 548/221; 548/193; 544/400

(57) ABSTRACT

Provided herein are compounds and methods of using compounds that selectively inhibit binding to one or more docking domain regions of an extracellular signal-recognition kinase to inhibit in a cell having an extracellular signal-regulated kinase activity. Such methods may be used to inhibit cell proliferation of a neoplastic cell, to treat a cancer and further may be used in conjunction with administration of an anticancer drug at a reduced dosage to treat a cancer with a concomitant reduction in toxicity to an individual receiving the treatment. Also provided is a method to design and screen for compounds to inhibit binding within the extracellular signal-regulated kinase docking domain region, using at least in part computer-aided drug design modeling.

### FEDERAL FUNDING LEGEND

This invention was produced in part using funds obtained through grants CA105299-01, CA95200-01 and CA095200-03S1 from the National Institutes of Health. Consequently, the federal government has certain rights in this invention.

### BACKGROUND OF THE INVENTION

1. Field of the Invention

The present invention relates generally to the fields of enzymology, computer-aided drug design and screening and oncology. More specifically, the present invention relates to specific inhibitors of extracellular signal-regulated kinase (ERK) docking domains useful in the treatment of cancer.

2. Description of the Related Art

Mitogen activated protein (MAP) kinases consist primarily of the extracellular signal regulated kinases 1 and 2 (ERK1/2), c-Jun N-terminal kinases (JNK), and p38 MAP kinases (1). MAP kinases play a central role in the regulation of most biological processes including cell growth, proliferation, differentiation, inflammatory responses and programmed cell death. Unregulated activation of MAP kinases has been linked to cancer cell proliferation and tissue inflammation (2-5).

Activation of ERK proteins most often occurs through a process where a ligand-activated plasma membrane receptor facilitates the sequential activation of the Ras G-proteins, Raf kinases, and the MAP or ERK kinases-1 and 2 (MEK1/2), which are the only known activators of ERK1 and ERK2 (7). The activation of ERK proteins by MEK1/2 is regulated by direct phosphorylation of threonine (Thr) 183 and tyrosine (Tyr) 185, where the amino acid numbering is according to mouse sequence, accession #P63085, where phosphorylation of both sites is required for full activation. Once ERK is phosphorylated, it undergoes structural changes that are important for phosphoryl transfer onto substrate proteins (8).

In vitro studies suggest that active ERK proteins may phosphorylate more than 50 different substrates (1,7). However, it is not clear whether all of these substrates are physiological targets in vivo or whether activated ERK selectively phosphorylates specific substrates in response to a particular extracellular signal. Importantly, hyper-activation of the ERK MAP kinases has been linked to unregulated cell proliferation in cancer cells. For example, naturally occurring mutations in Ras and Raf proteins, which cause hyper-activation of the ERK pathway, are found in almost 30% of all human cancers (3,9-10).

The mechanisms involved in determining the interactions between the ERK proteins and their cognate substrate proteins are still largely unknown. Similarly, it is not clear how ERK distinguishes between its own protein substrates and substrates that are phosphorylated by the JNK or p38 MAP kinases. Studies in recent years have revealed at least three protein motifs that provide clues as to how ERK proteins interact with and phosphorylate specific substrate proteins.

First, ERK proteins are proline directed serine or threonine kinases that prefer the consensus PXS/TP (X is any amino acid, P is proline, S is serine, and T is threonine) motif on the substrate protein (11). At a minimum, ERK proteins require a proline that is immediately C-terminal to the phosphorylated S or T residue. Second, ERK substrates may contain an FXFP (F is phenylalanine) motif, a D-domain containing basic residues followed by an LXL motif, or a kinase interaction motif (KIM), which are important for substrate interactions with ERK (12-13). Third, ERK proteins contain recently identified docking domains that have been shown to facilitate interactions with substrate proteins (14-16). The first identified ERK2 docking domains, referred to as common docking (CD) and ED domains, are positioned opposite the activation loop in the 3D crystallographic structure and appear to regulate the efficiency of substrate phosphorylation and interaction with the upstream MEK proteins (16). More recent data suggest that additional amino acid residues in the C-terminal domain of ERK2 may also form additional docking domains that regulate specific substrate interactions (14).

No specific inhibitors of the ERK proteins are currently available. Pharmacological inhibitors of Ras G-proteins, Raf kinases, and MEK1/2 have been used successfully to block the ERK pathway and are being tested in cancer clinical trials (17-20). Since ERK proteins are involved in many cellular functions, it may be more beneficial to selectively block ERK involvement in abnormal cell functions, such as cancer cell proliferation, while preserving ERK functions in regulating normal metabolic processes. Given that most kinase inhibitors lack specificity because they compete with ATP binding domains that are conserved among protein kinases (6,21), it is contemplated that small molecular weight compounds that interact with specific ERK docking domains can be used to specifically disrupt ERK2 interactions with protein substrates. Recent successes in CADD approaches in the identification of inhibitors of protein-protein interactions (23-26), indicated that such an approach was feasible for identification of inhibitors specific to ERK.

There is a need in the art for improvements in the development of specific small molecular weight MAP kinase inhibitors as an effective approach towards the identification of chemotherapeutic and anti-inflammatory agents. Specifically, the prior art is deficient in inhibitors that block extracellular signal-regulated kinase docking domains. The present invention fulfills this long-standing need and desire in the art.

### SUMMARY OF THE INVENTION

The present invention is directed to a method of inhibiting an activity of an extracellular signal-regulated kinase in a cell. The method comprises contacting the cell with an inhibitory compound that selectively binds to one or more docking domain regions of the ERK thereby inhibiting an ERK activity associated with an ERK substrate binding thereto.

The present invention also is directed to a method of inhibiting proliferation of a neoplastic cell. The neoplastic cell is contacted with an inhibitory compound that selectively inhibits binding of a substrate of an extracellular signal-regulated kinase to one or more docking domain regions thereof whereby proliferation of the neoplastic cell is inhibited. The inhibitory compound may be compound 17, compound 76, compound 89, compound 92, compound 93, or compound 95.

The present invention is directed further to a method of treating a cancer in a subject. The method comprises administering an inhibitory compound that selectively binds one or more docking domain regions of an extracellular signal-recognition kinase. Reducing proliferation of the cancer cells treats the cancer. The method may comprise a further step of administering an anticancer drug to the individual.

The present invention is directed to a related method of reducing toxicity of a cancer therapy in an individual in need thereof. The method comprises co-administering to the individual an inhibitory compound that selectively binds one or more docking domain regions of an extracellular signal-recognition kinase and an anticancer drug. The dosage of the anticancer drug administered with the inhibitor is lower than a dosage required when the anticancer drug is administered singly. Toxicity of the cancer therapy to the individual is thereby reduced.

The present invention is directed further still to a method of identifying an inhibitor of substrate binding to a docking domain region of an extracellular signal-reduction kinase. A test compound that binds one or more docking domain regions in the extracellular signal-regulated kinase, but does not interfere with the ATP binding domain, is designed based at least in part, computer-aided drug design (CADD) modeling. Inhibitory efficacy is determined by measuring the level of phosphorylation of a ERK substrate protein in the presence or absence of the test compound and comparing the level of protein phosphorylation in the presence of the test compound with the level of protein phosphorylation in the absence of the test compound. A decrease in protein phosphorylation in the presence of the test compound is indicative that the test compound is an inhibitor of binding to one or more docking domain regions in ERK.

The present invention is directed to a further method of screening the inhibitor for anti-cell proliferative activity directed against neoplastic cells. A culture of the neoplastic cells having an activated ERK activity is contacted with the inhibitor and the amount of cell proliferation of the neoplastic cells in the presence of the inhibitor is compared with the amount of cell proliferation of the neoplastic cells in the absence of the inhibitor. A decrease in cell proliferation in the presence of the inhibitor compared to cell proliferation in the absence of the inhibitor is indicative that the inhibitor has the ability to prevent cell proliferation in neoplastic cells.

The present invention is directed further still to inhibitory compounds identified by the screening methods described herein. These compounds inhibit binding one or more docking domain regions in ERK and thereby arrest proliferation of neoplastic cells. These compounds may be used in any of the methods of inhibiting cell proliferation of a neoplastic cell, of treating a cancer or of reducing toxicity of an anticancer drug described in the present invention.

The present invention is directed further to a related ERK inhibitory compound. The ERK inhibitory compound has a chemical structure comprising one or more substituted or unsubstituted heterocyclic aromatic ring moieties that are covalently coupled in a size and shape designed to bind one or more docking domain regions of an extracellular signal-reduction kinase without interfering with an ATP binding domain therein. The design of the synthetic compound is based at least in part on computer-aided drug design models. The present invention also is directed to a related ERK inhibitory compound. The substituted or unsubstituted heterocyclic aromatic ring moieties may be nitrogen, sulfur, or oxygen heteroatoms or a combination thereof and further have at least one of a pendant heteroatom, a pendant moiety having one or more heteroatoms, a side-chain having one or more heteroatoms or a combination thereof. The present invention also is directed further to the related ERK inhibitory compound that forms a bond with residues Asp316, Asp319 or a combination thereof comprising the CD domain and with at least one of residues Glu79, Asn80, Gln130, Arg133, Tyr314, Gln313 comprising the ED domain.

Other and further aspects, features, and advantages of the present invention will be apparent from the following description of the presently preferred embodiments of the invention. These embodiments are given for the purpose of disclosure.

### BRIEF DESCRIPTIONS OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1A-1B depict putative inhibitor binding sites on ERK2 and represent approximate orthogonal views of the protein. FIG. 1A shows the structure of the phosphorylated form of ERK2 with residues implicated in substrate interactions (yellow with residue number in black). Spheres demarcating putative binding pockets are shown in red for the S1 site, green for the S2 site, and white for the remaining sites (S3-S9). FIG. 1B shows the activation site residues Thr183 and Tyr185 (olive).

FIG. 2 depicts a ribbon diagram of the 3D structure of the unphosphorylated form of ERK2 showing the spatial relationship of the ERK2 phosphorylation sites and the docking region. Phosphorylation residues Thr183 and Tyr185 are red spheres, common docking (CD) residues Asp316 and Asp319 are blue spheres, and the ED residues Thr157 and Thr158 are green spheres. The putative docking groove is located between the CD and ED residues.

FIGS. 3A-3B show the molecular weight distribution of top compounds. FIG. 3A shows the molecular weight distributions of the top 20,000 compounds screened against unphosphorylated ERK2 based on normalized and unnormalized vdW attractive energies obtained during primary database screening. Distribution for the entire database is also shown. FIG. 3B shows the molecular weight distributions of the top 500 compounds based on normalized and unnormalized total interaction energies obtained during secondary screening.

FIGS. 4A-4C demonstrate the effects of test compounds on RSK-1 or ELK-1 phosphorylation. In FIGS. 4A-4B HeLa cells were treated with EGF for 5 minutes with or without 100 mM of test compounds. FIG. 4A shows an immunoblot of RSK-1 phosphorylated on Thr573 (pRSK-1). The far left lane is the control (−) with no EGF. The corresponding densitometry graph shows the relative pRSK-1 expression. The control (C) is EGF only treatment. In FIG. 4B cells pretreated with increasing concentrations of compound 76 were stimulated with EGF. ELK-1 phosphorylation on Ser383 (pELK-1) was measured by immunoblotting. The expression of dually phosphorylated ERK1/2 (ppERK1/2) and a-tubulin as a loading control are also shown. FIG. 4C demonstrates inhibition of ELK-1 phosphorylation by test compounds 86-98 targeting the phosphorylated (active) ERK2 protein. HeLa cells were pre-treated with 75 mM of the test compounds for 30 minutes followed by treatment with EGF for 5 minutes. Protein lysates were separated by SDS-PAGE and immunoblotted simultaneously for phosphorylated ELK-1 (pELK-1, Ser383), dually phosphorylated ERK1/2 (ppERK1/2), and a-tubulin as a protein loading control. Compounds 89, 92-93 and 95, and to a lesser degree, 94 inhibited EGF-mediated ELK-1 phosphorylation but had little effect on ERK1/2 phosphorylation. The corresponding densitometry graph shows the relative pELK expression. The control (C) is EGF only treatment.

FIGS. 5A-5B depict the structures of compounds tested in ERK substrate phosphorylation assays. Compounds 17, 36, 67, 68, 76, 79, 80, and 81 were identified using the unphosphorylated ERK2 protein structure (FIG. 5A). Compounds 86-98 were identified using the phosphorylated ERK2 protein structure (FIG. 5B).

FIGS. 6A-6C demonstrate the effect of test compounds on ERK2 fluorescence. The fluorescence (F) is plotted against the log concentration in moles/liter (Log [M]) for each compound is shown. ERK2 fluorescence in the absence of the test compound was set at 100%. Fluorescence titration of ERK2 was done with compounds 36, 67, 76, and 81 and compounds 17, 76 and 79-80 (FIG. 6A) identified using the unphosphorylated ERK2 protein structure. Fluorescence titrations were done using compounds 92-95 and compounds 86, 89 and 98 (FIG. 6B) identified using the phosphorylated ERK2 protein structure. FIG. 6C demonstrates that point mutations in the docking domains inhibit fluorescence quenching by compound 76. Fluorescence titrations of ERK2 wild type (WT) or ERK2 with T157A or D316N mutations were done with compound 76. The peak fluorescence values at 341 nm in the absence of 76 were 580 for ERK2 WT, 364 for T157A, and 599 for D316N.

FIGS. 7A-7B show predicted binding of active compounds to ERK2. The binding mode of 17 (FIG. 7A) or 76 (FIG. 7B) is shown. The ERK2 structure is shown in gray. The space-filling model of the docked compounds is predicted to form contacts with several amino acids within the groove between Asp316 and Asp319 of the CD domain (blue spheres) and Thr157 and Thr158 of the ED domain (green spheres). Sulfur, oxygen, or nitrogen atoms on the active compounds are indicated as yellow, red, or blue spheres, respectively.

FIGS. 8A-8C demonstrate inhibition of cell proliferation with test compounds. HeLa, A549, or SUM-159 cells were plated at a low density of 200-400 cells per well in the absence or presence of putative ERK docking domain inhibitors. Cell colonies were stained with crystal violet and counted after 6-10 days. In FIG. 8A cells were grown on 10 cm plates in the absence (−) or presence of 100 mM of compound 67, 36, 68, 81, or 76. Colony formation dose response in the presence of the indicated concentrations of compound 76 (closed squares) or 81 (open squares) for Hela cells (100 mM) (FIG. 8B), A549 cells (50 mM) (FIG. 8C), or SUM-159 cells (50 mM) (FIG. 8D). In FIG. 8E cells were grown on 1.5 cm wells in the absence (−) or presence of 0, 25, or 75 mM of compound 92, 94, or 95.

### DETAILED DESCRIPTION OF THE INVENTION

In one embodiment of the present invention there is provided a method of inhibiting an activity of an extracellular signal-regulated kinase (ERK) in a cell, comprising contacting the cell with an inhibitory compound that selectively binds to one or more docking domain regions of the ERK thereby inhibiting an ERK activity associated with an ERK substrate binding thereto.

In all aspects of this embodiment the extracellular signal-recognition kinase may be ERK1 or ERK2. Also, in all aspects the docking domain region comprises one or more of a CD domain, an ED domains, a SB domain, or a MS domain. Representative examples of the inhibitory compound are compound 17, compound 36, compound 76, compound 79, compounds 80-81, or compounds 92-95.

In an aspect of this embodiment the cell is a neoplastic cell. Examples of a neoplastic cell are those cells comprising a breast cancer, a lung cancer, a cervical cancer, a pancreatic cancer, a bladder cancer, a colon cancer, or a cancer having a Ras mutation.

In another embodiment of the present invention there is provided a method inhibiting proliferation of a neoplastic cell, comprising contacting the neoplastic cell with an inhibitory compound that selectively inhibits binding of a substrate of an extracellular signal-regulated kinase to one or more docking domain regions thereof whereby proliferation of the neoplastic cell is inhibited; wherein said inhibitory compound is compound 17, compound 76, compound 89, compound 92, compound 93, or compound 95. In all aspects of this embodiment, the extracellular signal-recognition kinases, the docking domains and the cancers are as described supra. In yet another embodiment of the present invention there is provided a method of treating a cancer in a subject, comprising administering an inhibitory compound that selectively binds to one or more docking domain regions of an extracellular signal-recognition kinase to reduce proliferation of cells comprising the cancer upon binding said inhibitory compound thereto, thereby treating the cancer in the subject.

Further to this embodiment the method may comprise administering an anticancer drug to the subject. In aspects of this embodiment, the anticancer drug may be administered concurrently or sequentially with the inhibitory compound. In another aspect of this embodiment a dosage of the anticancer drug is lower than a dosage required when the anticancer drug is administered singly, thereby reducing toxicity of the anticancer drug to the individual. Examples of anticancer drugs are cisplatin, oxaliplatin, carboplatin, doxorubicin, a camptothecin, paclitaxel, methotrexate, vinblastine, etoposide, docetaxel hydroxyurea, celecoxib, fluorouracil, busulfan, imatinib mesylate, alembuzumab, aldesleukin, and cyclophosphamide.

The inhibitory compounds may be compound 17, compound 36, compound 76, compound 79, compound 80, compound 81, or one of compounds 86-98. Preferably, the inhibitory compounds may be compound 17, compound 76, compound 89, compound 92, compound 93, or compound 95. Additionally, in all aspects of these embodiments the extracellular signal-recognition kinases, the docking domains and the cancers are as described supra.

In a related embodiment the present invention provides a method of reducing toxicity of a cancer therapy in an individual in need thereof, comprising administering to the individual an inhibitory compound that selectively binds to one or more docking domain regions of an extracellular signal-recognition kinase (ERK) and an anticancer drug, where a dosage of the anticancer drug administered with the inhibitory compound is lower than a dosage required when the anticancer drug is administered singly, thereby reducing toxicity of the cancer therapy to the individual. In aspects of this embodiment, the anticancer drug may be administered concurrently or sequentially with the inhibitory compound. In all aspects the extracellular signal-recognition kinases, the docking domains, the inhibitory compounds, the anticancer drugs and the cancers are as described supra.

In still another embodiment of the present invention there is provided a method of identifying an inhibitor of substrate binding to a docking domain region of an extracellular signal-reduction kinase (ERK), comprising designing a test compound that binds to one or more docking domain regions in ERK, but does not interfere with the ATP binding domain, wherein the design is based at least in part on computer-aided drug design (CADD) modeling; measuring the level of phosphorylation of an ERK substrate protein in the presence or absence of the test compound; and comparing the level of protein phosphorylation in the presence of the test compound with the level of protein phosphorylation in the absence of the test compound, wherein a decrease in protein phosphorylation in the presence of the test compound is indicative that the test compound is an inhibitor of binding to the docking domain region in ERK.

Further to this embodiment the method comprises screening the inhibitor for anti-cell proliferative activity directed against neoplastic cells. In this further embodiment screening comprises contacting a culture of the neoplastic cells having an activated ERK activity with the inhibitor; and comparing the amount of cell proliferation of the neoplastic cells in the presence of the inhibitor with the amount of cell proliferation of the neoplastic cells in the absence of the inhibitor, where a decrease in cell proliferation in the presence of the inhibitor compared to cell proliferation in the absence of the inhibitor is indicative that the inhibitory compound has the ability to prevent cell proliferation in neoplastic cells.

In all aspects of these embodiments the extracellular signal-recognition kinase may be ERK1 or ERK2. Also, in all aspects the docking domain region comprises one or more of a CD domain, an ED domains, a SB domain, or a MS domain. Further to this aspect the inhibitory compound may form a bond with residues Asp316, Asp319 or a combination thereof comprising the CD domain and with at least one of residues Glu79, Asn80, Gln130, Arg133, Tyr314, Gln313 comprising the ED domain. Again in all aspects the neoplastic cells and cancers are as described supra.

In a related embodiment there is provided an inhibitory compound identified by the methods of screening for an inhibitor of substrate binding to a docking domain region of an extracellular signal-reduction kinase (ERK) and of inhibiting cell proliferation of a neoplastic cell. In another related embodiment there is provided an ERK inhibitory compound having a chemical structure comprising one or more substituted or unsubstituted heterocyclic aromatic ring moieties covalently coupled in a size and shape designed to bind to one or more docking domain regions of an extracellular signal-reduction kinase without interfering with an ATP binding domain therein, said design based at least in part on computer-aided drug design models.

In all aspects of this embodiment the heterocyclic aromatic ring comprises nitrogen, sulfur, or oxygen heteroatoms or a combination thereof. In a particular aspect the substituted heterocyclic aromatic ring moieties comprise at least one of a pendant heteroatom, a pendant moiety having one or more heteroatoms, a side-chain having one or more heteroatoms or a combination thereof. Additionally, in all aspects the extracellular signal-reduction kinase is ERK1 or ERK2 and the docking domain region comprises one or more of a CD domain, an ED domains, a SB domain, or a MS domain. Further to these aspects the compound forms a bond with residues Asp316, Asp319 or a combination thereof comprising the CD domain and with at least one of residues Glu79, Asn80, Gln130, Arg133, Tyr314, Gln313 comprising the ED domain.

In a related embodiment there is provided an ERK inhibitory compound having a chemical structure comprising one or more substituted or unsubstituted heterocyclic aromatic ring moieties comprise nitrogen, sulfur, or oxygen heteroatoms or a combination thereof and further comprises at least one of a pendant heteroatom, a pendant moiety having one or more heteroatoms, a side-chain having one or more heteroatoms or a combination thereof covalently coupled in a size and shape, said substituted heterocyclic aromatic ring moieties designed to bind to one or more docking domain regions of an extracellular signal-reduction kinase without interfering with an ATP binding domain therein. In this embodiment the docking domain regions and the amino acid residues to which the ERK inhibitory compounds forms bonds are as described supra.

In a further related embodiment there is provided an ERK inhibitory compound having a chemical structure comprising one or more substituted or unsubstituted heterocyclic aromatic ring moieties comprising nitrogen, sulfur, or oxygen heteroatoms or a combination, and said substituted heterocyclic aromatic ring moieties comprises at least one of a pendant heteroatom, a pendant moiety having one or more heteroatoms, a side-chain having one or more heteroatoms or a combination thereof covalently coupled in a size and shape designed to bind within a CD or ED docking domain region of an extracellular signal-reduction kinase without interfering with an ATP binding domain therein, wherein said compound forms a bond with residues Asp316, Asp319 or a combination thereof comprising the CD domain and with at least one of residues Glu79, Asn80, Gln130, Arg133, Tyr314, Gln313 comprising the ED domain.

As used herein, the term, “a” or “an” may mean one or more. As used herein in the claim(s), when used in conjunction with the word “comprising”, the words “a” or “an” may mean one or more than one. As used herein “another” may mean at least a second or more. As used herein, the term “compound” or “inhibitor” or “inhibitory compound” means a molecular entity of natural, semi-synthetic or synthetic origin that blocks, stops, inhibits, and/or suppresses substrate interactions with ERK protein docking domains while not interfering with the ATP binding domain. As used herein, the term “heteroatom” or “heterocyclic” refers to an atom in an organic molecule or compound that is nitrogen, oxygen, sulfur, phosphorus or a halogen or an aromatic compound comprising the heteroatom. It is particularly contemplated that a heteroatom is nitrogen, oxygen or sulfur.

As used herein, the term “contacting” refers to any suitable method of bringing one or more of the compounds described herein or other inhibitory agent into contact with an ERK protein, as described, or a cell comprising the same. In vitro or ex vivo this is achieved by exposing the ERK protein or cells comprising the same to the compound or inhibitory agent in a suitable medium. For in vivo applications, any known method of administration is suitable as described herein.

As used herein, the term “neoplasm” refers to a mass of tissue or cells characterized by, inter alia, abnormal cell proliferation. The abnormal cell proliferation results in growth of these tissues or cells that exceeds and is uncoordinated with that of the normal tissues or cells and persists in the same excessive manner after the stimuli which evoked the change ceases or is removed. Neoplastic tissues or cells show a lack of structural organization and coordination relative to normal tissues or cells which usually results in a mass of tissues or cells which can be either benign or malignant. As would be apparent to one of ordinary skill in the art, the term “cancer” refers to a malignant neoplasm.

As used herein, the term “treating” or the phrase “treating a cancer” or “treating a neoplasm” includes, but is not limited to, halting the growth of the neoplasm or cancer, killing the neoplasm or cancer, or reducing the size of the neoplasm or cancer. Halting the growth refers to halting any increase in the size or the number of or size of the neoplastic or cancer cells or to halting the division of the neoplasm or the cancer cells. Reducing the size refers to reducing the size of the neoplasm or the cancer or the number of or size of the neoplastic or cancer cells.

As used herein, the term “subject” refers to any target of the treatment.

Provided herein are compounds that inhibit ERK protein docking domains by selectively blocking substrate interactions and methods of using these compounds to treat pathophysiological conditions having an unregulated cell proliferative component. By targeting unique regions on ERK, increased selectivity of these compounds in blocking ERK-specific phosphorylation of RSK-1 and ELK-1 may be achieved compared to typical kinase inhibitors that act as competitive inhibitors of ATP. The ERK proteins may target dozens of different substrates in vivo. Selective inhibition of substrates involved in unregulated cell proliferation may be achieved by targeting ERK docking domains. Computer-aided drug design (CADD) provides for the identification of compounds that disrupt ERK interactions with substrates involved in pathophysiological conditions while preserving ERK interactions with substrates needed for normal metabolic processes and cell maintenance.

Potential inhibitors of cell proliferation of cancer cells may be natural, semi-synthetic or synthetic compounds that have been designed or screened from chemical libraries or may be a synthetic derivative or analog compound having a structure similar to a known inhibitor. Inhibitors of ERK substrate docking identified by the methods described herein can block proliferation of cancer cells without affecting normal cell proliferation. Such inhibitors may be used to inhibit proliferation of neoplastic cells, to treat a cancer or to reduce the toxicity of a cancer drug to normal cells.

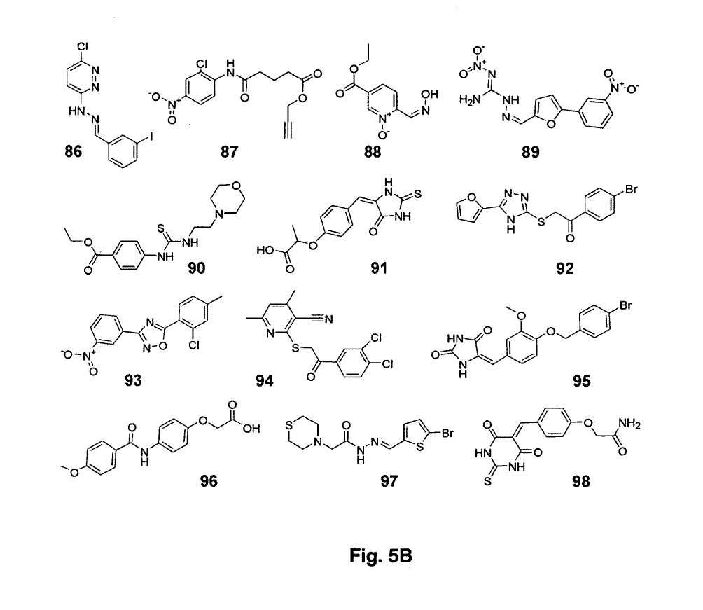

Accordingly, using the phosphorylated or unphosphorylated ERK2 crystal structure in a CADD (22) screening of a virtual database, small molecular weight compounds that disrupt ERK function by interacting with binding sites of one or more docking domain regions of ERK2 to selectively inhibit ERK-specific phosphorylation of substrates have been identified. Moreover, biological assays revealed that these lead compounds were effective in preventing proliferation of cancer cell lines. The inhibitory compounds so identified using unphosphorylated ERK2 include compound 17, compound 36, compound 76, compound 79, and compounds 80-81. The inhibitory compounds so identified using phosphorylated ERK2 include compounds 89-98. Preferably, compounds 17, 76, 89, 92-93, and 95 are useful as therapeutics. The structures are shown in FIGS. 5A-5B.

Potential inhibitory compounds identified by CADD modeling may be screened for inhibitory activity directed against substrate binding to ERK docking domain regions, for example CD, ED, SB, or MS. Without being limiting, for example, an inhibitory compound may inhibit substrate binding to the CD and ED docking domain region of ERK2. The inhibitory compound may block, stop, inhibit, and/or suppress substrate binding to one or more of these docking regions at one or more binding sites S1-S9 (see Table 2).

For example, ERK-associated phosphorylation activity may be assayed in the presence of ATP and a substrate phosphorylated via ERK and in the presence or absence of the potential inhibitor. A decrease in substrate phosphorylation in the presence of the potential inhibitor compared to substrate phosphorylation in the absence of the potential inhibitor is indicative that it has an ability to inhibit ERK substrate binding within the docking domain region of ERK. Such enzyme assays are known and standard in the art.

Subsequently, any potential inhibitor of the MLK-associated activity may be used in the cell proliferation assays. For example, a cancer cell culture having activated ERK activity is contacted with a potential inhibitory compound. A decrease in cell proliferation, as compared to control, may be determined by standard assays, such as a colony formation assay, trypan blue exclusion or other such assay known in the art.

It is contemplated that these compounds may be used as lead compounds from which other novel inhibitory compounds may be designed using at least the CADD modeling described herein. Predicted binding orientations of these compounds may be verified using X-ray crystallography or NMR spectroscopy, as is known in the art. Additionally, the CADD screen may be expanded to identify additional molecules that will act as lead compounds for the development of novel ERK inhibitors that can be used for experimental and clinical purposes. It also is contemplated that CADD may be applied to target docking domains of other MAP kinases, such as p38, in order to develop novel immunosuppressant agents.

For example the inhibitors may be synthetic compounds designed to have a chemical structure that at least includes one or more heterocyclic aromatic rings in the structure. These aromatic ring moieties are covalently coupled to be a size and shape to bind within the docking domain region of ERK without interfering with or inhibiting ATP binding to ERK. The heteroatoms comprising the rings may be one or more of nitrogen, sulfur, oxygen or a combination thereof. The aromatic ring moieties may be substituted or unsubstituted. Substituent atoms or molecules may be, but are not limited to, one or more of a pendant heteroatom, a pendant moiety having one or more heteroatoms, a side-chain having one or more heteroatoms or a combination thereof. The chemical structure is sufficient to form one or more ionic bonds and/or one or more pi bonds with residues from one or more of the CD, ED, SB, or MS domains. For example, an inhibitor may form a bond with Asp316, Asp319 or a combination thereof comprising the CD domain and with at least one of residues Glu79, Asn80, Gln130, Arg133, Tyr314, Gln313 comprising the ED domain. Generally, Table 2 in Example 2 provides a list of substrates and putative ERK2 docking domain sites with available residues.

The inhibitory compounds provided herein may be used to treat any subject, preferably a mammal, more preferably a human, having a pathophysiological condition characterized by the presence of transformed cells, e.g., a neoplasm, such as, but not limited, to a cancer. For example, a cancer may be a breast cancer, a lung cancer, a cervical cancer, a pancreatic cancer, a bladder cancer, a colon cancer, or another cancer having a Ras mutation. Administration of the inhibitory compound to a subject results in growth arrest of cancer cells without affecting the growth of a normal cell. Thus, cell proliferation is inhibited and a therapeutic effect, up to and including killing the cancer, is achieved thereby treating the cancer. It is contemplated that the compounds of the present invention may be used to inhibit proliferation of non-malignant neoplastic diseases and disorders.

Such an approach of selective inhibition of ERK substrates may also reduce toxicity to normal cells, which is observed with many of the current chemotherapies. An anticancer drug may be administered concurrently or sequentially with the compounds of the present invention. The effect of co-administration with an effective compound is to lower the dosage of the anticancer drug normally required that is known to have at least a minimal pharmacological or therapeutic effect against a cancer or cancer cell, for example, the dosage required to eliminate a cancer cell. Concomitantly, toxicity of the anticancer drug to normal cells, tissues and organs is reduced without reducing, ameliorating, eliminating or otherwise interfering with any cytotoxic, cytostatic, apoptotic or other killing or inhibitory therapeutic effect of the drug on the cancer cells.

The compounds and anticancer drugs can be administered independently, either systemically or locally, by any method standard in the art, for example, subcutaneously, intravenously, parenterally, intraperitoneally, intradermally, intramuscularly, topically, enterally, rectally, nasally, buccally, vaginally or by inhalation spray, by drug pump or contained within transdermal patch or an implant. Dosage formulations of these compounds and of the anti-cancer drugs may comprise conventional non-toxic, physiologically or pharmaceutically acceptable carriers or vehicles suitable for the method of administration.

The compounds and anticancer drugs or pharmaceutical compositions thereof may be administered independently one or more times to achieve, maintain or improve upon a therapeutic effect. It is well within the skill of an artisan to determine dosage or whether a suitable dosage of either or both of the inhibitory compound and anticancer drug comprises a single administered dose or multiple administered doses. An appropriate dosage depends on the subject's health, the progression or remission of the cancer, the route of administration and the formulation used.

The following example(s) are given for the purpose of illustrating various embodiments of the invention and are not meant to limit the present invention in any fashion.

### Example 1

### Cells and Reagents

HeLa (human cervical carcinoma), A549 (human lung carcinoma), HT1080 (human fibrosarcoma), or MDA-MB-468 (breast adenocarcinoma) cell lines were purchased from American Type Culture Collection (ATCC, Manassas, Va.). The estrogen receptor negative breast cancer cells, SUM-159, were obtained from the University of Michigan Human Breast Cancer Cell SUM-Lines. All cell lines were cultured in a complete medium consisting of Dulbecco's modified Eagle medium (DMEM) supplemented with 10% fetal bovine serum (FBS) and antibiotics (Penicillin, 100 U/ml; Streptomycin, 100 μg/ml) (Invitrogen, Carlsbad, Calif.). Epidermal growth factor (EGF) and phorbol 12-myristate 13-acetate (PMA) were purchased from Sigma (St. Louis, Mo.) and used at final concentrations of 50 ng/ml and 0.1 μM, respectively. Antibodies against phosphorylated Rsk-1 (pT573), Elk-1 (pS383), and ERK (pT183, pY185) were purchased from Cell Signaling Technologies (Woburn, Mass.), Santa Cruz Biotech. (Santa Cruz, Calif.), and Sigma, respectively. The α-tubulin antibody was purchased from Sigma.

### ERK Substrate Phosphorylation and Immunoblotting

Control and treated cells were washed twice with cold phosphate buffered saline (PBS, pH 7.2; Invitrogen) and proteins were collected following cell lysis with 300 μl of cold tissue lysis buffer (20 mM Tris, pH 7.4, 137 mM NaCl, 2 mM EDTA, 1% Triton X-100, 0.1% SDS, 25 mM P-glycerophosphate, 2 mM sodium pyrophosphate, 10% glycerol, 1 mM sodium orthovanadate, 1 mM phenylmethylsulfonyl fluoride (PMSF), 1 mM benzamidine), allowed to incubate on ice for about 10 minutes and then centrifuged at 20,000 (×g) to clarify the lysates of insoluble material. The lysates then were diluted with an equal volume of 2× SDS-sample buffer and the proteins were separated on SDS-PAGE for immunoblot analysis. Immunoblot analysis was done as previously described (41-43).

To expedite analysis of large numbers of samples from a diverse number of cell lines, protein lysates from control and treated cells are spotted onto nitrocellulose membrane using a Minifold-1 spot blot (96 well) apparatus (Whatman/Schleicher and Schuell). The nitrocellulose is sectioned into four quadrants each containing 24 spots. Each sample is spotted within each of these quadrants, which are cut and are immunoblotted with a specific antibody. Experiments will initially immunoblot the four sections of membrane using antibodies against pELK-1, pRSK-1, ppERK1/2, and α-tubulin. This method of analysis will only work with antibodies that have been shown to be specific for the protein of interest after SDS-PAGE and immunoblotting. The four antibodies mentioned fit these criteria. Quantification of the immunoblots will be done by densitometry (44). In addition, conditions, such as protein loading amounts and exposure times are established so that quantification is within the linear range of the densitometer.

An antibody microarray approach (45) is used to analyze the phosphorylation status of multiple substrates under control and treated conditions. This technology is current available through several vendors (eg. BD Biosciences). Proteins extracted from control and treated cells are labeled with fluorescent dyes (Cy3 and Cy5). The labeled proteins are then incubated with the antibody microarray containing a customized assortment of phosphorylation-specific antibodies against ERK-specific substrates. Table 1 lists some of the available phospho-specific antibodies against ERK substrates that are tested. The validation of antibody specificity first is done by SDS-PAGE and immunoblotting. In addition, the effects of substrates specific for the other major MAP kinases, JNK, and p38, are tested.

TABLE 1

| | Phosphorylation | |
|---|---|---|
| | sites | Company |
| ERK | | |
| RSK-1 | T359/S360/T573 | Cell Signaling |
| RSK-3 | T353/356 | ″ |
| ELK-1 | S383 | ″ |
| c-Myc | T58/S62 | ″ |
| MNK-1 | T197/T202 | ″ |
| PPAR-g | S112 | Chemicon |
| Tyrosine hydroxylase S31 | | ″ |
| Connexin-43 | S255 | Santa Cruz |
| Estrogen receptor-a | S118 | ″ |
| Tau | S199/S202 | Santa Cruz/BioSource |
| JNK | | |
| c-Jun | S63/S73 | Cell Signaling |
| p53 | T81 | ″ |
| p38 | | |
| ATF-2 | T71 | Cell Signaling |
| MAPKAPK-2 | T334 | ″ |
| MNK-1 | T197/T202 | ″ |
| Stat-1 | S727 | ″ |
| MSK-1 | S369/S376 | ″ |

### Colony Formation Assay

Two methods were used to determine cell proliferation and survival based on colony formation. First, HeLa cells were grown to 70-80% confluence and then treated for 16 hours in the absence (DMSO only) or presence of active compounds. The next day cells were trypsinized, replated (1000-2000 cells per 10 cm culture dish) in regular media and allowed to grow for 8-14 days. Cells then were fixed for 10 minutes in 4% paraformaldehyde and stained with 0.2% crystal violet (in 20% methanol) for 1-2 minutes. Cells were washed several times with distilled water and colonies formed of at least 40 cells were counted. In the second method, growing cells were trypsinized and replated (500-1000 cells per 35 or 60 mm well, respectively) in the presence or absence of various concentrations of the test compounds. Following incubation for 8-14 days, cells were fixed, stained with crystal violet, and counted as described above.

### Protein Purification

ERK2 was purified as described previously (46) with some modifications. Briefly, (His)₆-tagged ERK2 was expressed in bacteria and the cells were harvested in BugBuster protein extraction reagent (EMD Biosciences, San Diego, Calif.). Clarified lysates were loaded onto a Talon Co²⁺-IMAC affinity chromatography resin column (BD Biosciences, San Jose, Calif.) and the bound protein was eluted using increasing concentrations of imidazole. SDS-PAGE electrophoresis and Coomassie blue staining were used to identify the eluted fractions containing the ERK2 protein. The ERK2 protein concentration was determined using Bradford Reagent (Sigma). Phosphorylated ERK2 will be generated by dual phosphorylation on the Thr183 and Tyr185 active sites by incubation with a constitutively active MEK1 mutant as previously described (46).

### Fluorescence Titrations

Direct binding interactions between ERK2 and the biologically active compounds will be determined using fluorescence spectroscopy (47). Experiments will measure the changes in the intrinsic ERK2 fluorescence due to the presence of aromatic amino acids, with the indole group of tryptophan being the major fluorophore with absorption and emission maxima around 280 and 340 nanometers (nm), respectively. Fluorescence spectra were recorded with a Luminescence Spectrometer LS50 (Perkin Elmer, Boston, Mass.). For all experiments, ERK2 protein was diluted into 20 mM Tris-HCl, pH 7.5. Titrations were performed by increasing the test compound concentration while maintaining the ERK2 protein concentration at 3 mM. Unphosphorylated and phosphorylated ERK2 typically are incubated with 1, 5, 10, 25, 50, 75, or 100 mM of the biologically active compounds and the fluorescence intensity is measured. If necessary, higher inhibitor concentrations are used to saturate fluorescence quenching. The excitation wavelength was 295 nm and fluorescence was monitored from 300 to 500 nm. All reported fluorescence intensities are relative values and are not corrected for wavelength variations in detector response. Dissociation constants, K_{D}, were determined using reciprocal plots, 1/v vs 1/[I], where v represents the percent occupied sites calculated assuming fluorescence quenching to be directly proportion to the percentage of occupied binding sites, [1] represents the concentration of the inhibitor compound and the slope of the curve equals the K_{D} (48-49). Because the test compounds contain aromatic structures, the emission spectra of the active compounds in the absence of ERK2 will be determined. Based on the fluorescence of the active compounds in the absence of ERK2, the ERK2 fluorescence intensity changes will be corrected for compound fluorescence as required.

### X-Ray Crystallography

The unphosphorylated (His)₆-tagged ERK2 is expressed and purified as described above with additional purification through Mono Q and Phenyl Superose columns as previously described (50). Briefly, the purified protein is dialyzed against storage buffer (25 mM Tris-HCl [pH 7.4], 100 mM NaCl, 1 mM EDTA, and 0.1 mM DTT). Prior to crystallization, a 3-fold molar excess of the test compound is added to ERK2 (8 mg/ml) in storage buffer for 24 hr at 4° C. Crystals are grown in hanging drops at 16° C. by mixing 1 ml protein/peptide solution with 1 ml well solution containing 20% PEG 8000, 0.1 M sodium cacodylate, pH 7.0, and 0.2 M calcium acetate, and identified in Crystal Screen I (Hampton Research). Structure determination is done as previously reported (27,51).

### Pharmacokinetic Analysis

Tissue and plasma area under the curve from 0 to 24 hours (AUC₀₋₂₄) will be determined using Bailer's method (52). This method permits calculation of the variance associated with the AUC, thus yielding a 95% confidence interval (95% CI). Equation 1 will be used to calculate the AUC,

$$( {AUC}_{j} ) = {\sum }_{q = 1}^{m} {c}_{q} {\overline{y}}_{j} , q \qquad ( 1 )$$

where cq=1/2 D2 for q=1, 1/2 (Dq+Dq+1) for q=2 to q=m−1, cq=1/2 Dm for q=m; j is the number of groups, D is the time interval, m is the number of time points, and q is any given time point from 1 to m. y_{j,q} is the sample mean of the response at time q in group j. In our case number of groups j=1. The variance associated with the AUC was calculated using equation 2,

$${s}^{2} ( {AUC}_{j} ) = {\sum }_{q = 1}^{m} {c}_{q}^{2} [ \frac{{s}_{jq}^{2}}{{n}_{jq}} ] \qquad ( 2 )$$

where s²_{jq} is the variance associated with the response for each group at time point q, and n_{jq} is the number of animals per group at time point q. Clearance will be estimated for the Bailer calculated AUC by using equation 3.

$$Cl = \frac{Dose}{AUC} \qquad ( 3 )$$

The maximum concentration (Cₘₐₓ) and time of maximum concentration were the observed values. The drug exposure and pharmacokinetic parameters of maximum concentration (Cₘₐₓ), time of maximum concentration (tₘₐₓ), area under the concentration versus time curve (AUC), and terminal half-life (t_{1/2}) will be calculated compared between the treatment drugs.

### Example 2

### ERK2 Substrates and Putative Docking Domain Sites

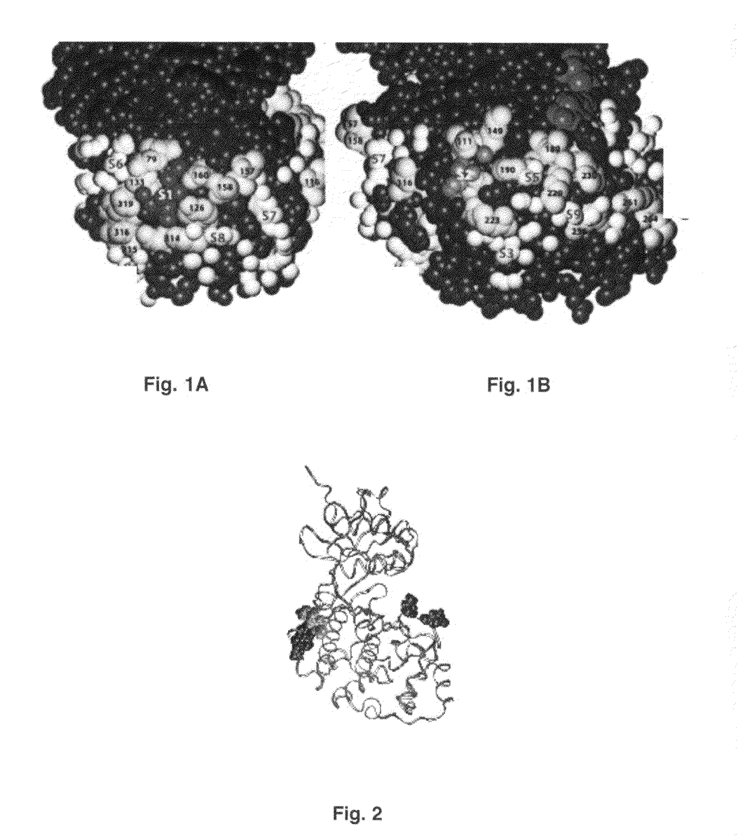

FIGS. 1A-1B show the residues that have been identified as being involved in ERK2-substrate interactions (14,34). As shown, a large number of residues may be involved in substrate interactions and these residues are distributed over a large region of the C-terminal portion of the protein. To identify novel putative binding sites in the vicinity of the substrate-binding residues, the program SPHGEN was used to identify concave regions on the entire protein surface and fill them with virtual spheres. Clusters of these spheres are used to direct the placement of ligands during virtual database screening as in Examples 3 and 4. Of the identified clusters, those with 5 or more spheres and with one or more spheres within 5 Å of any of the substrate-binding residues were identified and are shown as red, green, or white vdW spheres. Putative binding pockets, as defined by the sphere clusters, are identified as S1-S3.

Table 2 presents a summary of experimental data on ERK2 substrates and the ERK2 residues that interact with those substrates along with the associated putative binding sites that are shown in FIGS. 1A-1B. S1 originally was selected as it is adjacent to the common domain (CD) known to be important for the binding of a number of substrates and to the ED domain implicated in MEK1/2 and ELK-1 specific binding. The remaining sites were identified based on the density of the spheres in the clusters and their location relative to the residues of interest. It is contemplated that the ERK2 residues involved in MKP3 and MEK1 interactions also will be involved in regulating the efficiency of ERK interactions with other substrates.

TABLE 2

| | | | Putative | |
|---|---|---|---|---|
| | Site | | Binding | |
| Substrate | Name | Residues | Sites | Refs |
| Nonspecific | CD | Asp316, Asp319 | S1, S6 | (34) |
| Binding | | | | |
| MEK1 binding | ED | Thr157, Thr158 | S1, S7 | (14, 34) |
| ELK-1 | ED | Thr157, Thr158 | S1, S7 | (14, 34) |
| MKP3 binding | CD | Glu79, Tyr126, | S1, S6, S8 | (14, 16) |
| | | Arg133 Asp160, | | |
| | | Tyr314, Asp316, | | |
| | | Asp319 | | |
| MKP3 activation | SB | Tyr111, Thr116, | S2, S3, S5 | (14) |
| | | Leu119 Lys149, | | |
| | | Arg189, Trp190 | | |
| | | Glu218, Arg223, | | |
| | | Lys229 His230 | | |
| MEK1 binding | CD | Tyr315, Asp316, | S1, S6, S4 | (14) |
| | | Asp319, Asp320, | | |
| MEK1 binding | MS | His230, Asn236 | S9 | (53) |
| | | Tyr261, Ser264 | | |

S1: Between CD and ED domains, may impact MEK1/2 interactions and ELK-1, but lack of ERK substrate specificity is possible. S2: Between residues 111, 149, 190, 218, and 223; indicated to effect MKP3 activation. S3: Below 223; possible specificity for MKP3 activation; location on edge of identified residues may facilitate specificity. S4: Close to 315/316 implicated in MEK1 activation and binding, although 316 also implicated in MKP3 binding; location on edge of identified residues may facilitate specificity. S5: Between 189, 190, 223, 229, and 230 all involved in MKP3 activation. S6: In vicinity of 79, 133, 316, and 319 that are implicated for binding of a variety of substrates, may be general ERK substr te inhibitor. S7: Below 157/158 related to MEK1 and ELK-1 specificity; extended binding groove with decreased probability of having nonspecific effects associated with CD residues. S8: Close to 126 and 314 implicated in MKP3 activation; location on edge of identified residues may facilitate specificity. S9: Between 230 and 236 implicated in MEK1 specificity.

### Example 3

### General CADD Method for Compound Screening

Database Searching

The 3D structures of ERK2 in both the unphosphorylated and phosphorylated states (28,50) are available from the Protein DataBank (29). Charges and hydrogens are added to the proteins using SYBYL6.4 (Tripos, Inc.). All database searching calculations are carried out with DOCK 4.0.1, that includes in-house modifications, using flexible ligands based on the anchored search method (31). Ligand-protein interaction energies are approximated by the sum of the electrostatic and van der Waals (vdW, steric) components as calculated by the GRID method (35,54) implemented in DOCK using default values. In the GRID model a 3D lattice of hypothetical points is overlaid on the protein and the electrostatic and vdW potential due to the protein at each point is calculated. Interaction energies of ligands are then calculated based on the potential grid, rather than directly with the protein, yielding a significant saving in computer resources. The grid extends 15 Å beyond the respective sphere sets used for initial ligand placement in all dimensions, insuring that the docked compounds will be totally encompassed by the grid.

Identification of binding sites in the ERK2 docking domain is performed using the sphere sets calculated with the DOCK associated program SPHGEN. The solvent accessible surface (32) is calculated with the program DMS (33) using a surface density of 2.76 surface points per Å² and a probe radius of 1.4 Å² following which the spheres will be generated for the entire protein via SPHGEN. From the full sphere set, all sphere clusters with one or more spheres within 5 Å of any of the non-hydrogen atoms of residues experimentally identified to contribute to substrate binding are saved, as shown and discussed in Table 2 above.

Final selection of the putative binding sites for full docking are performed as follows. Each sphere cluster is analyzed individually, with individual spheres not part of the central region of the cluster manually deleted, thereby focusing the cluster. Preliminary docking is then performed against each cluster on 10,000 compounds, from which the binding response is calculated. The binding response is a modified scoring term that accounts for the spatial overlap of each docked compound with the sphere set such that if there is no overlap the binding response is 0 and if the overlap is ideal the value is 1, with the binding response for a particular binding site obtained by averaging over all 10,000 compounds. Visually, if the binding response is low, the docked compounds will be spread over a wide area around the binding site while in the case of a site with a binding response approaching one the compounds will be docked in a focused fashion overlaying the binding site. The binding response of each of the sites in Table 2 above are calculated with those sites with higher binding responses being prioritized.

Primary database searching is performed using the phosphorylated ERK2 3D structure on a 3D chemical database of over 3 million compounds. This includes commercially available compounds and compounds in the NCI 3D chemical database (55). The database has been compiled and converted from 2D structures to 3D structures (26,56).

Initiation of the database searches involves selection of compounds that contain 10 or less rotatable bonds and between 10 and 40 non-hydrogen atoms. Ligand flexibility is considered by dividing each compound into a collection of non-overlapping rigid segments, e.g. rings, referred to as anchors. Each anchor then is docked separately into the binding site in 200 different orientations, based on different overlap of the anchor atoms with the sphere set, and energy minimized. The remainder of each molecule is built onto the anchor in a stepwise fashion until the entire molecule is built, with each step corresponding to a rotatable bond. At each step the dihedral about the rotatable bond, which is connecting the new segment being added to the previously constructed portion of the molecule, is sampled in 10° increments and the lowest energy conformation is selected based on the interaction energy. During the build-up procedure selected conformers are removed based on energetic considerations and maximization of diversity of the conformations being sampled (37-38). The orientation of the compound with the most favorable interaction energy is finally selected.

From the initial DOCK runs, the top 50,000 compounds are selected based on normalized vdW attractive interactive energies. Use of the vdW attractive energy, versus total energy or electrostatic energy, forces the procedure to select compounds with structures that sterically complement the binding site. If electrostatics were included in the selection, compounds that did not fit the binding site well, but had strong favorable electrostatic interactions, i.e. ion pairs, would be chosen. The normalization procedure is designed to control the molecular weight (MW) of the selected compounds (46). Use of N^{1/2} normalization where N is the number of non-hydrogen atoms in the compounds, typically selects compounds with an average molecular weight of 320 daltons. Such compounds are smaller than the average molecular weight of pharmaceutically active compounds based on the World Drug index. The smaller molecular weight of the lead compounds allows the addition of functional groups during lead optimization efforts (57).

Secondary database searching of the top 50,000 compounds from each binding site is performed by applying a more rigorous secondary docking approach, termed method 2, which includes simultaneous energy minimization of the anchor during the iterative build-up procedure. In addition, method 2 docking is performed against both the phosphorylated and unphosphorylated ERK2 structures for each of the 50,000 compounds. The inclusion of two structures at this stage of docking partially accounts for the lack of receptor binding site flexibility during the database search. For each compound the most favorable score from the two ERK2 protein conformations is used for the final ranking. Scoring is based on the total interaction energy, as compounds dominated by electrostatic energies would have been eliminated during method 1 screening. Normalization is used again for selection of the desired molecular weight distribution.

From this procedure the top 1000 compounds are selected for chemical similarity clustering. In chemical similarity clustering, each compound is assigned a “fingerprint” based on the types of atoms in the compound and the connectivity between those atoms (e.g. atoms bonded to each other, atoms bonded to one of the atoms in the first bonded pair, and so on). The fingerprints of different compounds are then used to cluster the compounds into structurally similar sets based on the Tanimoto Similarity Index (39). This process yields approximately 100 clusters. One or two compounds are selected from each cluster for biological assay. This final selection process considers stability, potential toxicity, and solubility, where solubilities are estimated via calculated log P values using the Molecular Operating Environment (MOE, Chemical Computing Group). Selected compounds may be purchased from the appropriate vendor.

### Lead Validation

For an active compound to be considered a viable lead for additional studies it is ideal if it can be shown that the compound is a member of a class of active compounds. This may be performed by identifying compounds that are chemically similar to the active compounds based on the fingerprint analysis. Such an approach is similar to pharmacophore searching where it has been shown that compounds with similar structures should have similar biological activities (58). Application of this approach is necessary as the initial database search emphasizes chemical diversity during compound selection. In addition, with compounds that are active, but at decreased levels, identifying and assaying structurally similar compounds can identify compounds with enhanced activity, essentially rescuing low activity compounds and validating them as leads. It is contemplated that obtaining experimental data for collection of structurally similar compounds provides a basis for systematic structure-activity studies required for lead optimization.

Similarity searches targeting the 3 million compound database are performed as described. In these searches, the Tanimoto coefficient is adjusted to identify approximately 50 compounds for each active compound which are obtained for biological assay. These searches are performed following removal of extraneous substituents, e.g. methyl, amine or acid moieties, from the compounds that do not participate in linkers between ring systems. For molecules that contain three or more ring systems, similarity searches are done on analogs that contain only two rings. This approach allows for a wider variety of structurally similar compounds to be identified.

### Alternative Methods for DOCK Based Database Searching

DOCK based database searching makes a number of simplifications in order to minimize computer requirements, allowing for the databases of 3 million compounds to be searched. Of these simplifications the two most important are 1) the lack of conformational flexibility in the protein and 2) the simplified scoring function. If either of these assumptions becomes problematic, the following steps can be taken.

The assumption of a rigid protein during the docking procedure is necessary due to the large number of degrees of freedom in proteins, e.g., a conservative estimate is 10^{N}, where N is the number of amino acids. Two conformations of the ERK2 protein based on the crystal structures will be used in the method 2 search. If this number of conformations is deemed inadequate, additional conformations can be generated via molecular dynamics (MD) simulations of ERK2 in aqueous solution, using the molecular modeling program CHARMM (59-60). Molecular dynamics simulations will be performed to sample the conformational space of the putative binding sites described above. These additional conformations, typically 5, will be included in the method 2 search in addition to the crystal structures.

Alternate scoring methods will be attempted if significant improvements in the hit rates are not obtained. One alternate approach that may be applied with both method 1 and method 2 searches is consensus scoring (61-62). In this approach, several scoring functions are applied simultaneously, yielding improved estimation of the relative rankings of the docked compounds. This includes knowledge-based or potential of mean force (PMF) scoring methods that have been shown to yield improvements in the selection of correct orientations of ligands and have the advantage that they implicitly include certain aspects of solvation effects (63-64). Alternate approaches that may be used if deemed appropriate include generalized linear response methods (65-66) and free energy of solvation based on the Generalized Born (GB) model (67), including models included in the CHARMM program (68-69).

### Example 4

### Identification of Inhibitors of ERK2 S1 Binding Site in CD and ED

CADD In Silico Primary Screening Using Unphosphorylated ERK2

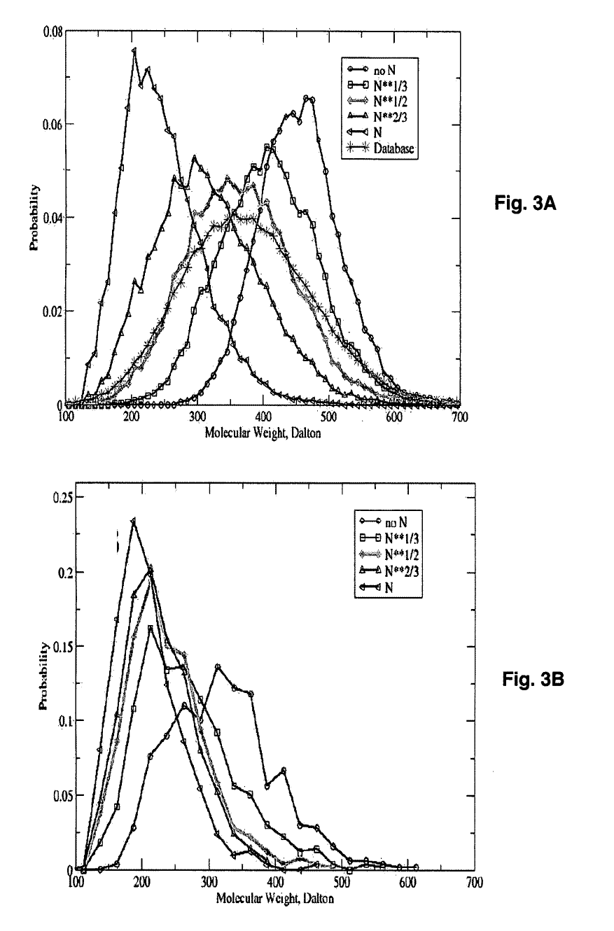

The ERK2 structure (FIG. 2) is bilobal in nature and is typical of many kinases where the amino and carboxyl lobes are separated by a hinge region (27). Upon phosphorylation of Thr183 and Tyr185 a conformational change brings the N-terminal lobe containing the ATP binding site in proximity to the C-terminal lobe to allow phosphate transfer onto substrate proteins. It has been suggested that substrate proteins interactions with ERK2 are determined by a common docking (CD) and ED domain regions in the C-terminus that interact with substrate binding motifs (14,34). This region was selected for the identification of putative binding sites, as inhibitors that bind to such sites will have the potential of blocking ERK2 substrate-protein interactions, with the inhibition potentially being specific for certain substrate proteins.

Sphere sets were calculated and sphere clusters in the region of the CD and ED docking domains in ERK2, which are important for interactions with the protein substrates, were identified. Based on mutagenesis experiments, residues involved in intermolecular interactions were used to select the docking site. These include Asp316 and Asp319 in the C-terminus (16), which are part of the common docking (CD) domain, and residues Thr157 and Thr158, which contribute to the ED docking domain (34). Spheres within both 10 Å of the CD domain and 12 Å of the ED domain were selected. The resulting sphere set contained 11 spheres and was located in the groove or cleft between the CD and ED domains as shown in FIG. 2. The GRID box dimensions were 25.3×26.6×27.3 Å³ centered around the sphere set to ensure that docked molecules were within the grid. The compounds that were screened had between 10 and 40 heavy atoms and less than 10 rotatable bonds.

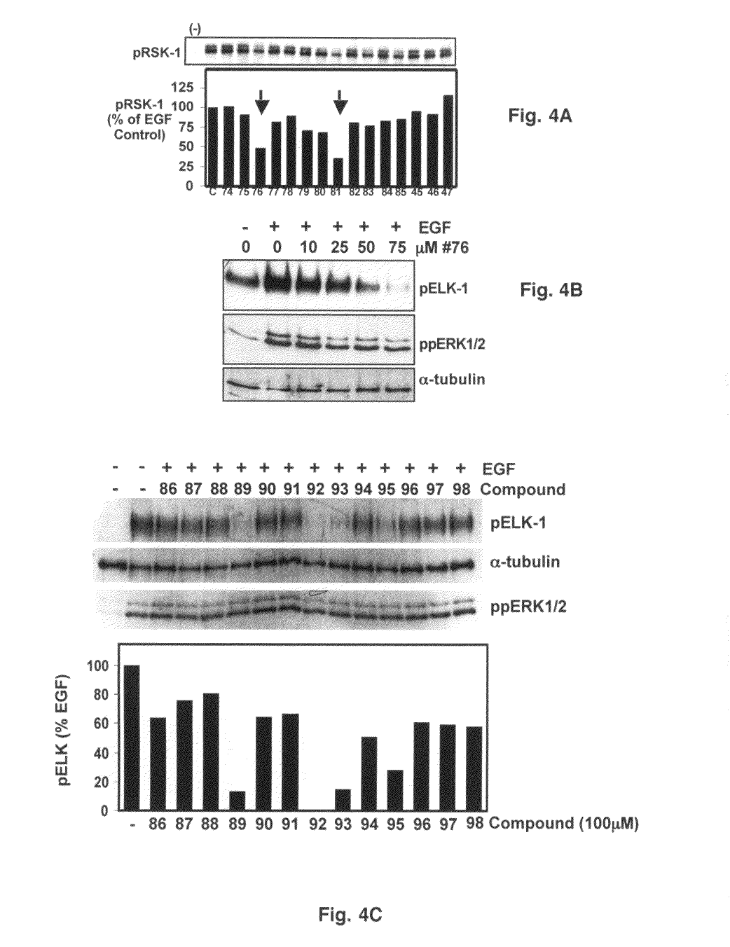

Use of the vdW attractive energy without any normalization yielded an average molecular weight for the top scoring compounds of 457 Da. This means that approximately half of those compounds are above a MW of 500 Da. As drug-like compounds typically have molecular weights below 500 Da (40) and the lead compounds have even lower molecular weights (70), it is desirable to select compounds with lower molecular weights via the normalization procedure. Using N, N^{2/3}, N^{1/2}, and N^{1/3} normalization the average molecular weights were 248, 317, 368, and 410 Da, respectively. FIG. 3A shows how larger powers of N shift the molecular weight distribution towards lower molecular weight values. To select the normalization procedure for compound selection it should be noted that the molecular weight probability distribution of the entire database screened in this Example is centered at 364 Da. Thus, N normalization was chosen since lead compounds of lower molecular weight are desired.

It should be noted that significant overlap of compounds occurs for the different normalization schemes. Of 20,000 compounds selected via N normalization, 11,355 compounds were common in the N^{2/3} set, 6540 in the N^{1/2} set, 3292 in the N^{1/3} set and 815 were in the set of non-normalized compounds. Thus, it may be assumed that compounds with highly favorable interaction orientations with the protein binding site are not being excluded by the normalization procedure.

### CADD In Silico Secondary Screening Using Unphosphorylated ERK2

After the primary screening, compounds were chosen for the secondary screening based on their normalized vdW attractive interaction energy scores. Compound selection based on the DOCK energy score favors compounds with higher molecular weight since their size contributes to the energy score. To minimize this size bias, an efficient procedure by which the DOCK energies are normalized by the number of heavy atoms N or by a power of N was applied as in Equation 4 (36):

IE_{norm,vdW}=IE_{vdW}/N^{x} (4).

Normalization of the vdW energies was done with x=1, 0.33, 0.5, and 0.67 and the molecular weight distributions of the top 20,000 compounds in each category were compared to the molecular weight of the database.

The total interaction energies of the top 20,000 compounds obtained in this Example were normalized and the molecular weight distributions of the top 500 compounds in each set using different powers of N were determined (FIG. 3B). For the top 500 compounds selected via the N, N^{2/3}, N^{1/2}, and N^{1/3} normalization, the average distributions were 210, 226, 238, and 267 Da, respectively. The average for the top 500 compounds without normalizing the energies was 321 Da. The top 500 scoring compounds in the set obtained after N^{1/3} normalization was chosen to avoid molecules which were too small, thereby lacking adequate structure diversity for lead or drug-like candidates.

Compounds 17, 36, 67-68, 76, and 79-81 selected via CADD were purchased from ChemDiv (San Diego, Calif.) or ChenBridge (San Diego, Calif.) and dissolved in DMSO at a stock concentration of 25, 50, or 100 mM. The purity of the active compounds was verified by mass spectrometry and thin-layer chromatography using 90% chloroform and 10% methanol as the solvent.

### CADD In Silico Secondary Screening Using Phosphorylated ERK2

This methodology, while successful, does not include the flexibility of the protein during docking. To partially account for this omission, the 20,000 compounds from the primary screen were docked against the 3D structure of the phosphorylated form of ERK2 using the secondary docking approach. From this second docking, 500 compounds were selected and compared with the original 500 compounds selected to target the unphosphorylated ERK2 structure. From this comparison, compounds unique to the second search were identified and subjected to cluster analysis to facilitate the selection of chemically dissimilar compounds. From this process a total of 45 novel compounds were selected to test in biological assays. Compounds 86-98 were obtained from commercial vendors and purified as described herein. Five compounds (89 and 92-95) have been shown to be active in ERK substrate phosphorylation assays.

### Example 5

### Compounds Effects on ERK Substrate Phosphorylation

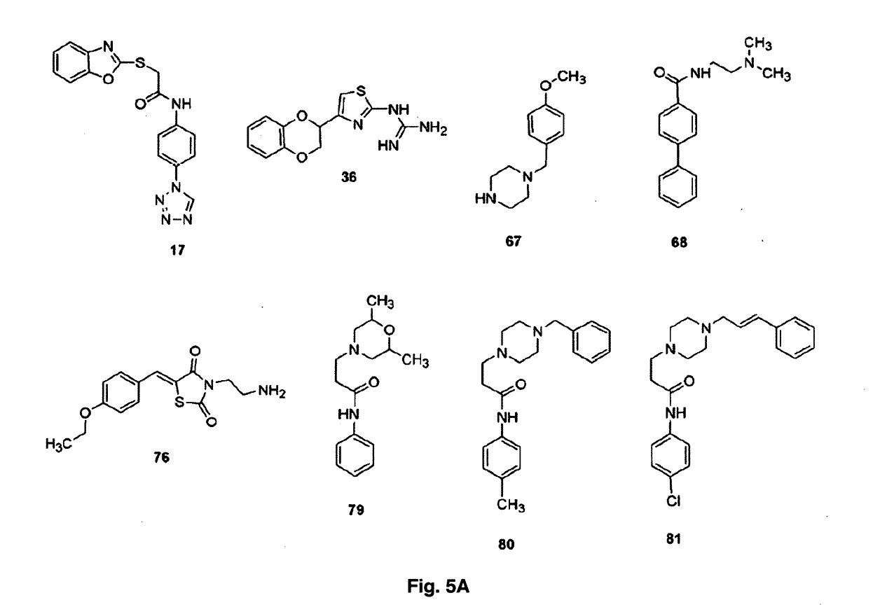

All obtained compounds were subjected to assays of ERK specific phosphorylation of Rsk-1 and/or Elk-1 as examined by immunoblot analysis using phosphorylation specific antibodies. In FIG. 4A HeLa cells were cultured in 24 well plates and pretreated for 20-30 minutes with 0-100 mM of the selected test compounds. The cells were then stimulated with epidermal growth factor (EGF, 50 ng/ml) for 5 minutes to activate the ERK pathway. Cell lysates were collected and immunoblotted for ERK-mediated phosphorylation of Rsk-1 on Thr573. As shown, EGF treatment alone caused a robust increase in Thr573 phosphorylation on Rsk-1 in the absence of test compounds. A typical immunoblot for Rsk-1 phosphorylation in the presence of 15 test compounds is shown in FIG. 4A. The presence of test compounds had varying inhibitory effects on ERK-mediated Rsk-1 phosphorylation. In these samples, densitometry quantification of the immunoblots showed that two compounds caused greater than 50% inhibition of Rsk-1 phosphorylation. Four additional compounds (17, 36, 79 and 80) inhibited ERK-mediated Rsk-1 phosphorylation by 20-25% out of the 80 compounds tested (data not shown).

The ERK-specific phosphorylation of the transcription factor Elk-1 on Ser383 was also tested with the compounds that showed the highest inhibition of Rsk-1 phosphorylation in FIG. 4A, i.e. compound 76. As shown, increasing doses of compound 76 inhibited ERK-mediated Elk-1 phosphorylation in response to EGF stimulation (FIG. 4B). As a protein loading control, the expression of α-tubulin was unchanged. Importantly, ERK1/2 phosphorylation on its activating sites was largely unaffected by the test compound. This finding support the specificity of this test compound for inhibiting ERK phosphorylation of downstream substrates, but has little effect on ERK protein phosphorylation by its upstream activator MEK1/2.

Compounds that were identified using the 3D structure of phosphorylated ERK2 also were tested. In FIG. 4C four (89, 92, 93, and 95) of the ten compounds tested showed evidence for inhibiting ERK-mediated ELK-1 phosphorylation. It should be noted that in vitro experiments using purified active ERK2 and a non-specific peptide substrate demonstrated that the test compounds did not affect ERK2 catalytic activity (data not shown). Therefore, these data suggest that CADD can identify compounds that will disrupt interactions with substrate proteins using either the unphosphorylated or phosphorylated ERK2 structures.

FIGS. 5A-5B show the chemical structures for some of the compounds that have been tested for their ability to inhibit ERK-mediated substrate phosphorylation. These include compounds 17, 36, 67, 68, 76, 79, 80, and 81, which were developed against the CD and ED domain (S1 site) using unphosphorylated ERK2 and compounds 86-98, which were developed against the S1 site using the phosphorylated (active) ERK2 protein structure. All compounds except compounds 36 and 68 showed some inhibition of ERK-mediated phosphorylation of RSK-1. Compound 36 was used as control as it had little effect on ERK substrate phosphorylation. The structure of compound 68 was included because it appeared to enhance ERK phosphorylation of RSK-1.

As shown, the compounds have diverse chemical structures, although some similarities are evident. For example, 17, 79, 80 and 81 have amide moieties directly adjacent to aromatic rings with many of compounds including piperazine groups. The advantage of having chemically diverse structures as this stage of the project is, during future lead optimization efforts, to maximize the potential that one or more of the compounds will have the desired bioavailability properties as well as specifically targeting ERK-substrate interactions.

### Example 6

### Fluorescence Titrations

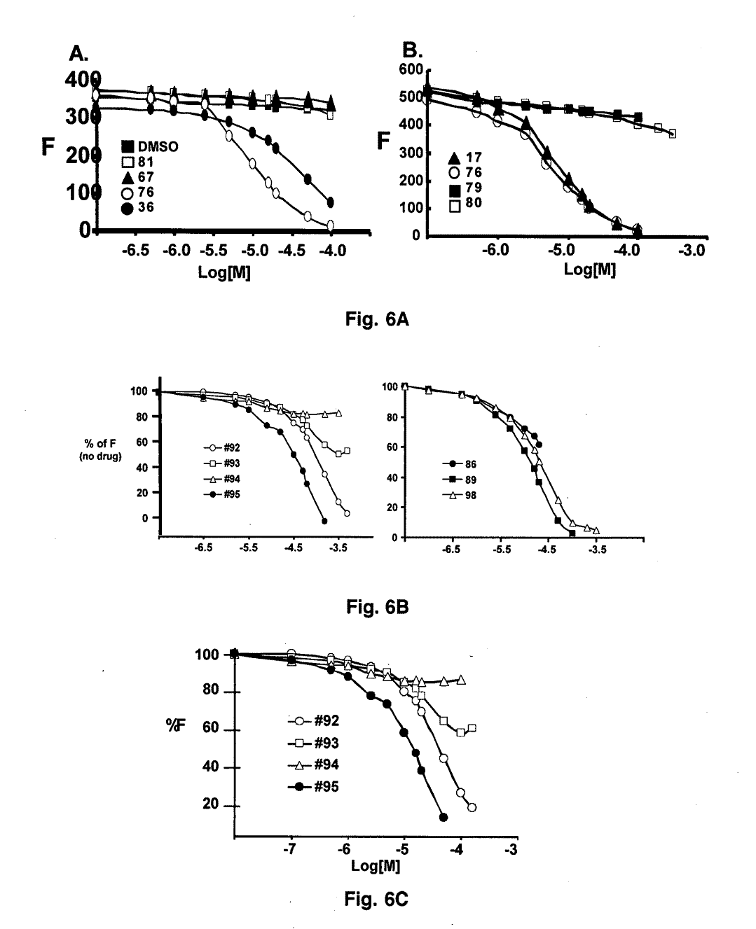

It was determined whether the active compounds directly interact with ERK2 using fluorescence spectroscopy. ERK2 contains three tryptophans, which have intrinsic fluorescence. Quenching of this fluorescence by the test compounds strongly suggest direct interaction between the compound and ERK2. Of the two compounds shown to be most active in all biological assays, 76 and 81, 76 shows strong quenching of fluorescence while quenching only occurs to a small extent at the higher concentrations with 81 (FIG. 6A).

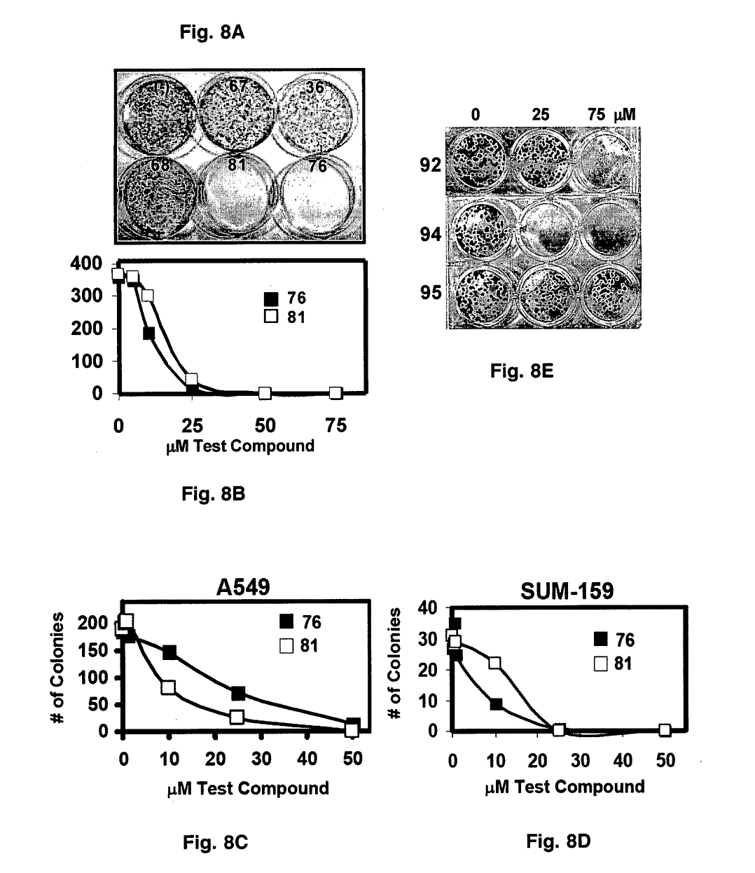

Compound 36 also showed significant quenching (FIG. 6A). Interestingly, compound 36, which also had little effect on ERK-mediated Rsk-1 phosphorylation but caused a subtle inhibition of colony formation (FIG. 8A below), showed significant binding with ERK2 (FIG. 6A). It is contemplated that compound 36 may be useful for future analysis of ERK function and substrate phosphorylation. In addition, compound 67, which significantly reduced RSK-1 phosphorylation also did not show quenching at the concentrations tested (FIG. 6A). Compound 68, which enhanced ERK-mediated RSK-1 phosphorylation showed strong auto-fluorescence in the absence of ERK2 protein. Thus, these assays could not determine whether compound 68 was interacting with ERK2. X-ray crystallography, as discussed in Example 1, may help determine the interactions between compounds that auto-fluoresce and ERK2.

From the fluorescence titrations, via reciprocal plots, K_{D} values of 5 and 16 mM were calculated for 76 and 36, respectively, with y-intercepts of 1.8 and 1.1, respectively, indicating a single binding site on the protein. Thus, the fluorescence quenching experiments indicate that 76 is binding directly to ERK2, thereby leading to its biological activity. Importantly, the K_{D} for compound 76, as determined from the fluorescence quenching, is similar to the approximate IC₅₀ determined based on colony formation (FIG. 8A below). Compound 17, which also inhibited colony formation, had a similar K_{D} as 76 (FIG. 6A). These findings suggest that any biological effects of 17, 36, and 76 are ERK-mediated while the effects of compounds 67, 79, 80, and 81 on ERK phosphorylation may not be via ERK-specific interactions.

The effects on ERK2 fluorescence were tested using the compounds that were identified to disrupt substrate interactions with the CD and ED domain using the phosphorylated ERK2 structure. As shown, compounds 86, 89, 92, and 95 quenched ERK2 fluorescence indicative of binding (FIG. 6B). Compounds 89 and 95 appeared to be more effective in quenching indicating a stronger affinity to bind ERK2. In contrast, compound 93 was less effective in interacting with ERK2 and appeared to auto-fluoresce at higher concentrations (FIG. 6B). X-ray crystallography as described in Example 1 may be used to determine compound 93 interactions with ERK2. Lastly, compound 94 did not appear to interact with ERK2. Thus, the effects of 94 on ERK substrate phosphorylation (FIG. 3C) may be non-specific. These data further support the identification of three compounds (89, 92 and 95), which bind to ERK2 and affect ERK-mediated substrate phosphorylation. As with compound 68, the mode of action for 93 must be determined.

To determine whether the compounds bind specifically to the region identified by CADD point mutations were generated in the CD or ED regions of ERK2 and tested whether compound binding to ERK2 via fluorescence titrations is altered. Both point mutations, Thr157 to alanine (T157A) in the ED domain and Asp316 to asparagine (D316N) in the CD domain, tested with compound 76 showed an approximately 5 fold reduction in binding affinity based on fluorescence quenching as compared to wild type ERK2 (FIG. 6C). These data indicate that changes in the binding pocket targeted by CADD disrupt compound binding providing evidence that the compounds are targeting the region of the ED and CD domain.

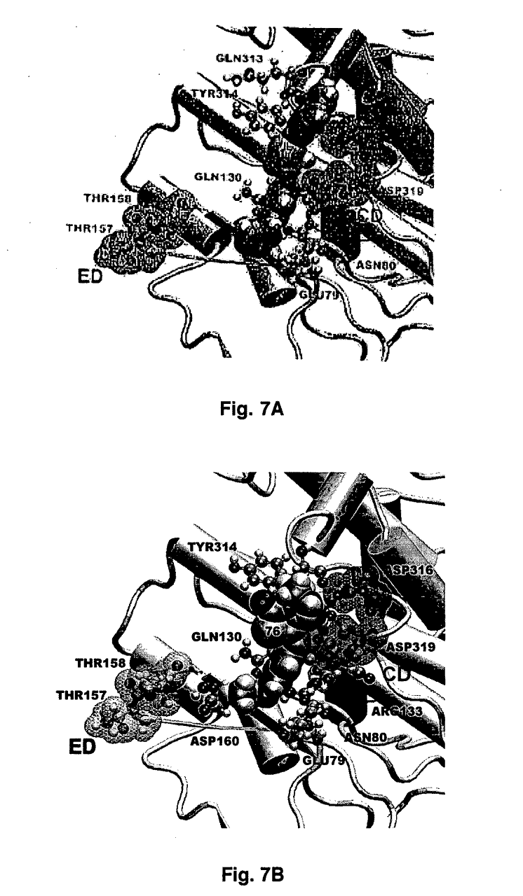

Alternatively, other amino acids depicted in FIGS. 7A-7B can be mutated using site directed mutagenesis (71) to characterize the CD and ED domain. Additional ERK2 mutants, containing a threonine to alanine mutation at residue 158, and an aspartate to alanine mutation at residue 319, may be generated. Moreover, ERK2 mutants at the other docking sites listed in Table 2 can be generated depending on the outcome of the CADD and substrate phosphorylation analysis. The fluorescence intensity is determined using ERK2 mutants incubated with the active compounds at the concentrations described above and compared with the fluorescence intensity determined using wild type ERK2. If an amino acid residue is important for the structure of a particular docking groove and binding of the test compounds, then fluorescence quenching will be diminished in the ERK2 mutants as compared to ERK2 wild type. To control for the possibility that docking site mutations may cause structural changes that affect catalytic activity, mutant and wild type ERK2 enzymatic activity will be compared in cells and in vitro.

### Example 7

### Predicted Structures of Ligand-ERK2 Complexes

As the experimental fluorescence results confirm that compounds 17 and 76 bind to directly ERK2, it is of interest to understand the nature of the interactions between those compounds and ERK2. A detailed atomic picture of the predicted binding modes for these compounds identified from the screen using the unphosphorylated ERK2, as described in Example 4, is presented in FIGS. 7A-7B. Based on these predicted binding conformations, the compounds fit nicely into the groove that is located between the ED and CD sites. With both compounds, binding is predicted to occur adjacent to the CD site which places the compounds approximately 5-7 Å away from the threonine residues of the ED site, which forms a small protrusion on the protein surface.

The groove into which the compounds bind is polar containing several charged amino acids that are involved in multiple favorable interactions with the compounds. ERK2 residues with atoms within 3 Å of the compounds were Glu79, Asn80, Gln130, Arg133, Tyr314, Gln313, and the two aspartates from the CD site, Asp316 and Asp319. Several hydrogen bonds are observed between the aspartates and 17 and 76 (FIGS. 7A-7B). Arg133 is located above the aromatic rings in 76 and 17 potentially forming a cation-pi bond. Tyr314 makes a CH . . . O interaction through its backbone oxygen with 76. In addition, if the protein structure was allowed to relax around the bound compound, it is likely that more inhibitor-ERK2 interactions would be identified. Thus, based on the predicted binding interactions, a number of inhibitor-protein interactions may contribute to both the binding affinity and the specificity for the ERK2 protein.

### Example 8

### Effects of Active Compounds on Cell Proliferation

The effects of the active compounds on cell proliferation and survival were tested using a colony formation assay. A screen of five test compounds showed that two compounds (76 and 81) completely inhibited cell proliferation, as evident by decreased number of cell colonies (FIG. 8A). Other compounds, including 36, 67, and 68, had little effect on colony formation (FIG. 8A).

Dose response assays demonstrated that compounds 76 and 81 similarly inhibited HeLa cell colony formation with an IC₅₀ of approximately 15-20 μM (FIG. 8B). In A549 lung carcinoma cells the IC₅₀ for compounds 76 and 81 was approximately 25 and 15 μM, respectively (FIG. 8C). Moreover, inhibition of cell proliferation following incubation with compounds 76 and 81 was observed in the SUM-159 estrogen-receptor negative breast cancer cell line (FIG. 8D) and HT1080 fibrosarcoma cells (data not shown). Compounds 17, 79, and 80 also inhibited HeLa, A549, HT1080, and MDA-MB-468 cell proliferation with IC₅₀ values similar to 76 and 81. Thus, several test compounds that show maximal inhibition of ERK substrates are also effective inhibitors of proliferation in cultured cancer cell lines.

Compounds 86-98 also were tested in the colony formation assay. Colony formation inhibition is shown for compounds 92 and 94-95. All compounds showed some degree of colony formation inhibition (FIG. 8E), although compound 94 (FIG. 8E) and 93 were the most effective inhibitors of colony formation, their effect may be non-specific as these compounds interactions with ERK2 were inconclusive as determined by fluorescence titrations (FIG. 6B). However, 92 and 95 inhibited in the 10-100 mM range, consistent with the binding in the fluorescence quenching experiment (FIG. 6B), indicating their function to be associated with direct binding to ERK. The differences in effects of active compounds on cell proliferation may be due to differences in how the active compounds affect ERK substrate phosphorylation. For example, active compounds that show stronger inhibition of cell proliferation may target a broader range of ERK substrates. Table 3 provides the IC₅₀ concentrations (micromolar) for compounds 86-98.

TABLE 3

| Compound | IC₅₀(μM) |
|---|---|
| 86 | 3-5 |
| 87 | >100 |
| 88 | >100 |
| 89 | <2 |
| 90 | ND |
| 91 | ND |
| 92 | ~75 |
| 93 | 5-10 |
| 94 | 5-10 |
| 95 | >100 |
| 96 | >100 |
| 97 | >100 |
| 98 | >100 |

### Example 9

### Effects of Test Compounds on JNK and p38 MAP Kinase Substrates

The effects of test compounds on the JNK- or p38-specific substrates are tested. Table 1 above includes some of the available phospho-specific antibodies against INK and p38 substrates. Since the docking domains that are targeted in ERK2 may share features with the p38 MAP kinases (34), it is determined whether the biologically active compounds target substrates that can be phosphorylated by both kinases. As one example, ERK and p38 dually phosphorylate the MAP kinase integrating kinase-1 (MNK-1) on the same threonine sites at positions 197 and 202 (72). Similarly, JNK and p38 may also target S383 on ELK-1. Compounds are tested for specificity to ERK, JNK or p38 substrate phosphorylation by treating cells with factors known to specifically activate each pathway. Cells are treated with epidermal growth factor (EGF) or anisomycin to activate ERK or p38, respectively. JNK activity can be specifically activated by over-expression of MLK3 (45). This determines whether the active compounds can selectively discriminate between the various MAP kinase substrates.

ERK or p38 activity in the context of treatment with candidate compounds is examined. HeLa cells are transfected with constitutively active mutants of MEK1, which only activates ERK proteins (73) or MEK3, which primarily activates p38 but not ERK (74). Transfected cells are incubated in the absence or presence of biologically active compounds and ERK2 or p38 substrate phosphorylation is determined by immunoblotting. It is contemplated that biologically active compounds that target p38 may have additional utility for the development of new molecules aimed at treating inflammatory diseases (75).

### Example 10

### In Vitro Experiments with Active ERK Incubated with Specific Substrates

In vitro kinase assays are done using purified active ERK2 (commercially available or generated as described in Example 1) incubated with specific substrates, ELK-1 and c-Myc (generated by expression vectors, for example) or a non-specific myelin basic protein (MBP) peptide in the presence of 0, 1, 5, 10, 20, 30, 40, 50, and 75 mM of the test compounds showing biological activity. The MBP peptide, which does not require the CD or ED domain in order to be phosphorylated by ERK, is used as a control for measuring the effects of the test compounds on ERK2 catalytic activity. ELK-1 or c-Myc substrate phosphorylation is measured by phosphorimager analysis following gel electrophoresis and expressed as a ratio of the MBP phosphorylation under each test drug concentration.

Although data (not shown) suggest that ERK activity is not affected by the test compounds, cell based experiments are performed to confirm these observations. HeLa cells are stimulated with EGF in the presence or absence of test compounds (0, 25, 50 or 100 mM) and ERK2 is immunoprecipitated for kinase assays done in the presence of radiolabeled ATP and the non-specific substrate MBP. MBP phosphorylation will be measured by scintillation counting. It is contemplated that immunoprecipitated EGF-stimulated ERK2 phosphorylation of MBP is not affected in the presence of the test compounds. As a control for the cell based and in vitro experiments, the general kinase inhibitor, staurosporine, is used to inhibit ERK2 activity.

Alternatively, the efficacy of the test compounds, identified using the ERK2 structure, for binding to ERK1 is determined using fluorescence titration assays. Whereas, the corresponding residues surrounding the ED domain are identical in ERK1 and ERK2, the CD domain is different as shown in the sequences below of the amino acids surrounding the CD domain region of ERK2 and the corresponding region in ERK1. The underlined amino acids are different between ERK1 and ERK2 in the CD domain

ERK2: P YLEQ YYD₃₁₆PSD₃₁₉EPI AEA (SEQ ID NO: 1)

ERK1: P YLEQ YYD₃₃₆PTD₃₃₉EPVAEE (SEQ ID NO: 2)

It is recognized that the test compounds may have effects on other MAP kinases, which are less well characterized. For example, chemical inhibitors of MEK1/2 may also inhibit the activity of the MEK5/ERK5 signaling pathway (76). In addition, consideration must be given to other kinases that are not related to MAP kinases but also play a role in the survival of cancer cells. For example, the serine/threonine kinase Akt has been implicated in promoting cancer cell survival (77). Once a candidate compound is identified, a comprehensive examination of its effects on multiple families of kinases are conducted using the antibody microarray in Example 1.

### Example 11

### Pharmacokinetics of Test Compounds

The cellular metabolism of the test drugs will be assessed in the HeLa, MDA-MB-468, SUM159, HCT116, and SK-Me1-28 cell lines. Experiments will first test compound 76, but it is anticipated that other compounds characterized in aims 1 and 2 will also be tested. The cellular metabolic profile and kinetics of test compound will be determined using the cells in vitro. To determine if changes in intracellular test compound and metabolite concentrations are relevant, the intratumoral pharmacokinetics of the test compound and its metabolites will be assessed in tumor bearing mice as in Example 1.

The test compounds and metabolites are quantified using a high performance liquid chromatography (HPLC) assay with ultraviolet (UV), fluorescence detection, or tandem mass spectrometry detection (LC/MS/MS) (78-80). Metabolite identification is verified using a modified liquid chromatography with triple quadrupole mass spectrometric detection (LC/MS/MS). After trypsinization, cells are plated onto 6 cm plates at a seeding density of a million cells per plate. After 24 hours, the cells are incubated with the test compounds at a concentration of 10-100 mM for 0, 5, 10, 30, or 60 minutes. The cells are harvested using 1N KOH and analyzed. Cellular uptake and kinetics of test compounds and major metabolites are measured in the cell lines using HPLC methods.

### Example 12

### In Vivo Tumor Model

Human MDA-MB-468 breast cancer cells are initially used as a xenograft model in athymic nude mice (nu/nu, 5-6 week old: Harlan Sprague Dawley, Inc.). These cells are well established for developing tumors in this model. However, HCT116 and SK-Me1-28 have also been shown to cause tumors in nude mice and may be used.

The hind leg is an established model for establishing a xenograft whose growth parameters may be measured and modeled to existing data. The nude mice are implanted subcutaneously with 10⁶ MDA-MB-468 cells in 0.5 ml sterile saline as described (18) in the presence or absence of test compounds. Tumor growth is monitored daily by calipers. Alternatively, after tumors reach a mean diameter of 4-5 mm² in size, the animals are left untreated or treated with the test compounds prepared in a vehicle of 10% DMSO/saline as below. Statistical comparisons are made using a two sample student's T test to assess the mean difference in tumor size between the control and treated groups. Significance is defined as difference in tumor size resulting in a p value <0.05.

At the beginning of cancer cell injection or after the tumors reach a mean diameter of 4-5 mm² in size, the animals are intravenously injected via the lateral tail vein with the test compounds (50 mg/kg) or vehicle control once daily for two days. Three animals per time-point are sacrificed at each of the following timepoints: 0.25, 1, 4, and 8 hours after test compound administration. Control blood and tissue for all study groups is collected at approximately 0.25 hours after administration of vehicle alone.

Blood is collected via cardiac puncture from three animals at each timepoint. The blood is centrifuged immediately at 4° C. at 1250×g for 10 minutes. Plasma is separated into cryotubes and stored at −80° C. until analysis. Organs (liver, heart, brain, kidney, and tumor tissue) are removed and placed immediately on dry ice, weighed and snap frozen in liquid nitrogen. All tissue is stored at −80° C. until time of analysis. The total number of animals for this study is 120. An additional 12 animals are used as untreated controls. The concentration of each test compound and its metabolites are measured via HPLC in mouse plasma, liver, heart, brain, kidney, and tumor tissue. Pharmacokinetic analysis is performed using a model independent approach as described in Example 1.

The following references are cited herein.
1. Pearson et al., 2001, Endocr Rev, 22:153-183.
2. Kyriakis, J. M. and Avruch, J., 1996, Bioessays, 18:567-577.
3. Reuteretal. 2000, Blood, 96:1655-1669.
4. Duesbery et al., 1999, Nat Med 1999, 5:736-737.
5. Shapiro, P. 2002, Crit Rev Clin Lab Sci 2002, 39:285-330.
6. Cohen, P., 1999, Curr Opin Chem Biol 1999, 3:459-465.
7. Lewis et al., 1998, Advances in Cancer Research 1998, 74:49-139.
8. Zhang et al., 1995, Structure 1995, 3:299-307.
9. Bos J. L., 1989, Cancer Res 1989, 49:4682-4689.
10. Brose et al., 2002, Cancer Res 2002, 62:6997-7000.
11. Songyang et al., 1996, Mol Cell Biol, 16:6486-6493.
12. Zuniga et al., 1999, J Biol Chem, 274:21900-21907.
13. Fantz et al., 2001, J Biol Chem, 276:27256-27265.
14. Zhang et al., 2003, J Biol Chem, 278:29901-29912.
15. Nichols et al., 2000, J Biol Chem, 275:24613-24621.
16. Tanoue et al., 2000, Nat Cell Biol, 2:110-116.
17. Bollag et al., 2003, Curr Opin Investig Drugs, 4:1436-1441.
18. Boldt et al., 2004, Curr Pharm Des, 10:1885-1905.
19. Sebolt-Leopold, J. S., 2004, Curr Pharm Des, 10:1907-1914.
20. Allen et al., 2003, Semin Oncol, 30:105-116.
21. Bain et al., 2003, Biochem J, 371:199-204.
22. Gohlke et al., 2002, Angew Chem Int Ed Engl, 41:2644-2676.
23. Markowitz et al., 2004, J Med Chem, 47:5085-5093.
24. Loughney et al., 1999, Med. Chem. Res., 9:579-591.
25. Zeng et al., 2001, Protein Eng, 14:39-45.
26. Huang et al., 2004, J Med Chem, 47:3502-3511.
27. Zhang et al., 1994, Nature, 367:704-711.
28. Canagarajah et al., 1997, Cell, 90:859-869.
29. Bernstein et al., 1977, Eur J Biochem, 80:319-324.
30. Kuntz et al., 1982, J Mol Biol, 161:269-288.
31. Kuntz, I. D., 1992, Science, 257:1078-1082.
32. Connolly, M. L., 1983, Science, 221:709-713.
33. Ferrin et al., 1988, J. Mol. Graphics, 6:13-27.
34. Tanoue et al., 2001, Embo J, 20:466-479.
35. Goodford et al., 1984, J. Med. Chem., 28:849-857.
36. Pan et al., 2003, J Chem Inf Comput Sci, 43:267-272.
37. Leach et al., 1992, J. Comput. Chem., 13:730-748.
38. Ewing et al., 1997, J. Comput. Chem., 18:1175-1189.
39. Butina et al., 1999, J. Chem. Inf. Comp. Sci., 39:747-750.
40. Lipinski et al., 2001, Adv Drug Deliv Rev, 46:3-26.
41. Cha et al., 2001, J Biol Chem, 276:48494-48501.
42. Dangi et al., 2003, Cell Signal, 15:667-675.
43. Cha et al., 2004, Biochem J, 378:857-865.
44. Dangi, S., and Shapiro, P., 2005, J Biol Chem, 280:24524-24531
45. Chan et al., 2004, Nat Med, 10:1390-1396
46. Shapiro et al., 1999 Mol Cell Biol, 19:3551-3560.
47. Lakowicz, J. R., Principles of Fluorescence Spectroscopy. Kluwer Academic/Plenum Publishers Second Edition 1999.
48. Marshall, G., Biophysical Chemistry: Principles, Techniques, and Applications. John Wiley & Sons: New York 1978.
49. MacKerell et al., 1991, Biochim Biophys Acta, 1073:357-365.
50. Chang et al., 2002, Mol Cell, 9:1241-1249.
51. Zhang et al., 1993, Journal of Molecular Biology 233:550-552.
52. Bailer, A. J., 1988, J Pharmacokinet Biopharm, 16:303-309.
53. Robinson et al., 2002, J Biol chem, 277:5094-5100.
54. Meng et al., 1994, J Comput Aided Mol Des 8:299-306.
55. Milne et al., 1994, J Chem Inf Comput Sci, 34:1219-1224.
56. Hayashi, J., and MacKerell, A. D., Jr., 2001, Immunomodulatory Compounds, USA.
57. Teague et al., 1999, Angew Chem Int Ed Engl, 38:3743-3748.
58. Nicklaus et al., 1997, J Med Chem, 40:920-929.
59. MacKerel, et al., 1998, Encyclopedia of Computational Chem. 1:271-277.
60. Brooks et al., 1983, J. Comput. Chem. 4:187-217.
61. Bissantz et al., 2000, J Med Chem, 43:4759-4767.
62. Charifson et al., 1999, J Med Chem, 42:5100-5109.
63. Gohike, H., and Klebe, G., 2001, Curr Opin Struct Biol, 11:231-235.
64. Muegge, I., and Martin, Y. C., 1999, J Med Chem, 42:791-804.
65. Chen, X., and Tropsha, A., 1999, J. Comput. Chem., 20:749-759.
66. Aqvist et al., 1994, Protein Eng, 7:385-391.
67. Qui et al., 1997, J. Phys. Chem., 101:3005-3014.
68. Lee et al., 2003, J Comput Chem, 24:1348-1356.
69. Feig et al., 2004, J Comput Chem, 25:265-284.
70. Oprea, et al., 2001, J Chem Inf Comput Sci, 41:1308-1315.
71. Cha, H., and Shapiro, P., 2001, J Cell Biol, 153:1355-1368.
72. Waskiewicz et al., 1997, Embo J, 16:1909-1920.
73. Mansour et al., 1996, Cell Growth Differ, 7:243-250.
74. Raingeaud et al., 1996, Mol Cell Biol, 16:1247-1255.
75. Herlaar, E., and Brown, Z., 1999, Mol Med Today, 5:439-447.
76. Mody et al., 2001, FEBS Lett, 502:21-24.
77. Datta et al., 1999, Genes Dev, 13:2905-2927.
78. Simmons et al., 1997, Analytical Letters, 30:2009-2021.
79. Rudek et al., 2000, J Pharm Biomed Anal, 22:1003-1014.
80. Bauer et al., 2000, Biomed Chromatogr, 14:338-343.

Any patents or publications mentioned in this specification are indicative of the levels of those skilled in the art to which the invention pertains. Further, these patents and publications are incorporated by reference herein to the same extent as if each individual publication was specifically and individually incorporated by reference.

One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

1-37. (canceled)

38. A synthetic compound having a chemical structure comprising:
one or more substituted or unsubstituted heterocyclic aromatic ring moieties covalently coupled in a size and shape designed to bind to one or more docking domain regions of an extracellular signal-reduction kinase without interfering with an ATP binding domain therein, said design based at least in part on computer-aided drug design models.

39. The synthetic compound of claim 38, wherein said heterocyclic aromatic ring comprises nitrogen, sulfur, or oxygen heteroatoms or a combination thereof.

40. The synthetic compound of claim 38, wherein one or more of said substituted heterocyclic aromatic ring moieties comprises at least one of a pendant heteroatom, a pendant moiety having one or more heteroatoms, a side-chain having one or more heteroatoms or a combination thereof.

41. The synthetic compound of claim 38, wherein said extracellular signal-reduction kinase is ERK1 or ERK2.

42. The synthetic compound of claim 38, wherein said docking domain region(s) comprises one or more of a CD domain, an ED domains, a SB domain, or a MS domain.

43. The synthetic compound of claim 42, wherein said compound forms a bond with residues Asp316, Asp319 or a combination thereof comprising the CD domain and with at least one of residues Glu79, Asn80, Gln130, Arg133, Tyr314, Gln313 comprising the ED domain.

44. An extracellular signal-regulated kinase inhibitory compound having a chemical structure comprising one or more substituted or unsubstituted heterocyclic aromatic ring moieties comprise nitrogen, sulfur, or oxygen heteroatoms or a combination thereof and further comprises at least one of a pendant heteroatom, a pendant moiety having one or more heteroatoms, a side-chain having one or more heteroatoms or a combination thereof covalently coupled in a size and shape, said substituted heterocyclic aromatic ring moieties designed to bind to one or more docking domain regions of an extracellular signal-reduction kinase without interfering with an ATP binding domain therein.

45. The extracellular signal-regulated kinase inhibitory compound of claim 44, wherein said docking domain region(s) comprises one or more of a CD domain, an ED domains, a SB domain, or a MS domain.

46. The extracellular signal-regulated kinase inhibitory compound of claim 44, wherein said compound binds with residues Asp316, Asp319 or a combination thereof comprising the CD domain and with at least one of residues Glu79, Asn80, Gln130, Arg133, Tyr314, Gln313 comprising the ED domain.

47. An extracellular signal-regulated kinase inhibitory compound having a chemical structure comprising one or more substituted or unsubstituted heterocyclic aromatic ring moieties comprising nitrogen, sulfur, or oxygen heteroatoms or a combination, and said substituted heterocyclic aromatic ring moieties comprises at least one of a pendant heteroatom, a pendant moiety having one or more heteroatoms, a side-chain having one or more heteroatoms or a combination thereof covalently coupled in a size and shape designed to bind within a CD or ED docking domain region of an extracellular signal-reduction kinase without interfering with an ATP binding domain therein, wherein said compound forms a bond with residues Asp316, Asp319 or a combination thereof comprising the CD domain and with at least one of residues Glu79, Asn80, Gln130, Arg133, Tyr314, Gln313 comprising the ED domain.


SEQUENCE LISTING (2 sequences)

SEQ ID NO: 1 - protein, 17 aa, artificial sequence
  CD domain of ERK1
PYLEQYYDPSDEPIAEA

SEQ ID NO: 2 - protein, 17 aa, artificial sequence
  CD domain of ERK2
PYLEQYYDPSDEPIAEA